(19) United States
(12) Patent Application Publication
(10) Pub. No.: US 2017/0150957 A9
(43) Pub. Date: Jun. 1, 2017

(54) INFLATABLE LAPAROSCOPIC RETRACTOR FOR ATRAUMATIC RETRACTION IN ABDOMINAL SURGERY

(71) Applicant: UNIVERSITY COLLEGE CORK, NATIONAL UNIVERSITY OF IRELAND, CORK, Cork, Co. Cork (IE)

(72) Inventors: Conor O'Shea, Cork (IE); Padraig Cantillon-Murphy, Cork (IE); Emmet Andrews, Cork (IE); Michael O'Riordain, Cork (IE); Conor O'Donoghue, Cork (IE); Grace Burke, Cork (IE); Daniel Cummins, Cork (IE); Richard Finn, Cork (IE)

(21) Appl. No.: 14/654,508

(22) PCT Filed: Dec. 20, 2013
(86) PCT No.: PCT/EP2013/077720

§ 371 Date: Jun. 19, 2015

(30) Foreign Application Priority Data
Dec. 21, 2012 (EP) 12198842.2

(51) Int. Cl. A61B 17/02

(52) U.S. Cl. CPC A61B 17/0218; A61B 2017/00314

(57) ABSTRACT

A laparoscopic retractor (11) comprises a shaft (8) having a proximal and distal end, the distal end of the shaft being configured for adjustment from a substantially straight orientation to a curved orientation having a substantially U-shaped hook suitable for laparoscopic retraction. The proximal end of the shaft comprises a handle (1) operably connected to the distal end of the shaft for adjustment of the distal end from the substantially straight orientation to the curved orientation. The distal end of the shaft is enclosed within a flexible sheath (4) that is adapted to be inflated, and wherein a portion of the sheath covering the distal end of the shaft comprises inflatable balloon means such as one or more inflatable balloons (5) which cover the substantially U-shaped hook.

### TECHNICAL FIELD

The present invention relates to a minimally invasive, deployable retractor for the use in laparoscopic surgery.

### BACKGROUND TO THE INVENTION

Laparoscopic surgery, also known as minimally invasive surgery, is a technique that allows surgery to be performed without the long traditional incision associated with open surgery. By using multiple small incisions and placing a surgical cannula or trocar through these incisions, the surgeon may manipulate the internal organs extracorporeally. The short term benefits of laparoscopic surgery include reduced hospital stay, less postoperative pain, earlier return to normal activity, improved cosmesis, and overall reduction in health-care costs. Laparoscopic lower gastrointestinal surgery is performed with the patient under general endotracheal anaesthesia. Once a pneumoperitoneum has been established, a number of surgical cannulas are inserted through the abdominal wall and the patient is placed into a Trendelenburg position.

One of the most common challenges encountered during laparoscopy is that of the distended loops of bowel or overlaying organs spilling into the operating field and thus obstructing the surgeon's view. Overcoming this requires changing the position of the patient and the table as well as tedious pushing or pulling of the intrusive intestines using a bowel graspers or a laparoscopic retractor. By placing the patient in the Trendelenburg position, which involves inclination of the patient's body with his or her head down and legs elevated, allows the small bowel to glide away from the pelvis, creating a working space within the abdominal cavity. To optimise surgical exposure, an angle of inclination of up to 40° may be required. However, prolonged Trendelenburg position significantly increases intracranial pressure and intraocular pressure. In addition to the circulatory effects, steep head-down impacts on the cardiac system due to increased central venous pressure, and on the respiratory system by decreasing total lung volume, pulmonary compliance, and functional residual capacity.

A number of retraction devices have been developed to assist in identifying the plane of dissection. Bowel graspers are the most common instrument used to manipulate and retain the internal organs. The surgeon relies on the haptic feedback from the graspers to delicately manipulate the internal organs. However, this feedback is severely limited due to the mechanical friction losses and variations in the transmission of forces over the working range. Furthermore, the relatively small tips on the laparoscopic graspers can generate high pressures locally on the soft tissue, which may lead to injury or perforation. Conversely insufficient force will lead to tissue slipping out of the graspers.

Alternative retractors are inserted into the body in a collapsed state, deployed within the body and either held by an assistant or may be fixed to the operating table. One such example is that of U.S. Pat. No. 5,656,012 which discloses a surgical retractor with a handle, elongated body, and a membrane extending from the distal end of the body supported by a pair of resilient bands. An actuation mechanism in the handle alternates the bands between an open and closed position. Once open, the membrane forms a paddle shape to manually manipulate the internal organs.

Another example is that of U.S. Pat. No. 5,439,476 which discloses an inflatable laparoscopic retractor with an inflatable paddle located on the distal end of a manually operated rod. After insertion, a balloon on the distal end of the rod is inflated providing a relatively wide palmated anterior surface which creates a soft interface between the balloon and internal organs. These devices offer advantages over the bowel graspers, namely limiting the possibility of injuring the organs, but they remain cumbersome and require a dedicated skilled operator to manoeuvre and handle the device over the duration of its use.

A third example of a laparoscopic retractor is presented with hinged links along a shaft that can be manipulated into a curved profile (U.S. Pat. No. 6,248,062). This solution enhances the manoeuvrability of the device and is primarily targeted at liver retraction. However, the possibility of pinching the soft tissue between its links when engaged makes the device unsuitable for bowel retraction. In addition, the contact area between the shaft and the organ is quite narrow.

In recent years, the use of robotic-controlled surgery has been proposed and clinically demonstrated. The da Vinci Surgical System by Intuitive Surgical of Sunnydale, Calif. is an example of such a system. It is proposed that the inflatable retractor detailed in this invention may be adapted to use with such a system whereby articulation of the device shaft, inflation and deflation of the balloons and insertion and withdrawal of the device from the peritoneal cavity can be controlled by a robotic system.

U.S. Pat. No. 5,571,115 describes a laparoscopic retractor having a hinged arm mounted on a distal end of the shaft, in which part of the hinged arm is covered in an inflatable balloon. The part of the device employed for laparoscopic retraction is L-shaped, and thus not ideal for laparoscopic retraction. Further, while some embodiments of the device include an inflatable balloon, the balloon is mounted on straight parts of the shaft. In addition, there is a risk of tissue being pinched in the hinge during operation of the hinged arm.

It is an object of the invention to overcome at least one of the above-referenced problems.

### SUMMARY OF INVENTION

The invention provides a laparoscopic retractor comprising a handle and a shaft, the shaft having a distal end and a proximal end, in which the distal end can be adjusted from a substantially straight orientation to a substantially U-shaped (curved) orientation, which orientation is ideal for laparoscopic retraction by hooking partially around the target organ(s). The distal end of the shaft is covered in a flexible sheath comprising inflatable balloon in fluid communication with the sheath such that when inflated the balloon means covers the distal end of the shaft. There are a number of advantages associated with this arrangement: first, the inflatable balloon on the curved portion of the shaft provides a greater surface area for engagement with the organ(s) to be retracted; secondly, the use of inflatable balloon means on the curved distal end cushions the engagement between the curved part of the shaft and the organ (s), thereby minimising risk of the movable parts of the shaft causing damage to the internal organs during adjustment; and thirdly, the use of a flexible sheath which embraces the distal end of the shaft and which carries the inflatable balloon means obviates the requirement for additional inflation lumens for the balloon means.

Accordingly, the invention provides a laparoscopic retractor comprising a shaft having a proximal and distal end, the distal end of the shaft being configured for adjustment from a substantially straight orientation to a curved orientation, ideally a U-shaped curved orientation, suitable for laparoscopic retraction. The proximal end of the shaft comprises a handle operably connected to the distal end of the shaft for adjustment of the distal end from the substantially straight orientation to the curved orientation, wherein the distal end of the shaft is enclosed within a flexible sheath that is adapted to be inflated, and wherein a portion of the flexible sheath covering the distal end of the shaft comprises inflatable balloon means.

In the curved orientation, the distal end of the shaft comprises a substantially U-shaped hook suitable for laparoscopic retraction. The balloon means extends along the length of the substantially U-shaped hook. The balloon means may be a single elongated balloon, or a plurality of balloons, arranged axially along the sheath. Typically, the balloon or balloons embrace the shaft. Providing the inflatable balloon means in the form of a plurality of balloons helps avoid kinking or undue deformation of the balloon means which can occur when a single balloon is provided.

Preferably, the balloon means covers the length of the curved portion of the distal end of the shaft. The or each balloon is generally tubular when inflated, and typically has a length of 60-250 mm, and a diameter (when inflated) of preferably 10-60 mm.

Preferably, the portion of the flexible sheath covering the distal end of the shaft comprises a plurality of inflatable balloons, for example at least 2 but no more than 6, and ideally 3 or 4. Generally, the or each balloon is a long spherical balloon, although other shapes are possible, for example spherical balloons or square balloons. Typically, the balloons are arranged in series, although they may also be arranged in parallel, especially when the balloons are spherical balloons. The balloons are typically inflated through a common inflation lumen, although the or each balloon may also be inflated through their own individual inflation channel.

Typically, the flexible sheath extends along the distal part of the shaft and also along at least part, or preferably all, of the proximal part of the shaft.

One embodiment of the or each balloon is a cylindrical profile whereby its shape is circular in cross section having a diameter of between 10 and 60 mm and its length can range from 20 to 250 mm. The corner shape of the balloon may be tapered, curved or square. The finishing surface of the balloons may be rough or dimpled to improve friction between the device and the internal organs and tissue.

A second embodiment of the or each balloon is an elliptic cylindrical profile whereby its shape is elliptical in cross section having a major and minor axis of between 10 and 60 mm and its length can range from 20 to 250 mm. The corner shape of the balloon may be tapered, curved or square.

A third embodiment of the or each balloon is that the balloon(s) shape is hammock-like whereby there are two long spherical balloons attached together at the distal and proximal ends and separated in the middle by a soft flexible mesh. The advantage of this type of balloon shape is the improved capturing of the internal organs especially the intestines.

A fourth embodiment of the or each balloon is a single elastomeric balloon capable of inflating at predetermined points along its length. This single balloon may have varying wall thickness's along the length of the balloon which will allow it to expand radially where desired.

The flexible sheath is typically formed of a polymeric material, for example silicone or polyurethane. This sheath acts as a platform to weld or otherwise affix a series of balloons in a linear arrangement near the distal end of the sheath. The proximal end of the sheath typically connects to the 1 handle and a cap seals a distal end of the sheath preventing leakage once intracorporeally positioned. An advantage of the balloon means is that they provide a soft interface between the device and the internal organs and tissue as well as increasing the contact surface area. This removes the risk of injuring the internal organs. The number of balloons may be as few as one but will not exceed six depending on their individual dimensions and the curvature of the device. The sheath has a central lumen for receiving the distal end of the shaft, and optionally a second inflation lumen in fluid communication with at least one balloon and means for inflating the balloons. The flexible sheath may also comprise a co-axial configuration whereby the gap between the co-axial sheath acts as an inflation channel for at least one balloon. Alternatively, the sheath may comprise a single lumen that receives the shaft and functions as the inflation lumen for the at least one balloon. The distal end of the extruded sheath along with its attached balloons is capable of curving with the central shaft once engaged by the control handle.

In one embodiment, the or each balloon is separately formed and attached to the sheath, for example by welding. In another embodiment, the or each balloon is unitary with the sheath.

Generally, the proximal end of the sheath comprises an inflation lumen adapted to engage with the inflation means. The inflation lumen generally branches off the sheath at a position adjacent the proximal end of the shaft. The proximal end of the inflation lumen is generally adapted to attach to an inflation means, for example a syringe, via a luer lock connection.

In one embodiment of the invention, the handle comprises an inflation actuator for inflation of the balloon means. Thus, the proximal end of the inflation lumen may be coupled to the inflation actuation as opposed to an external pump or a luer. The inflation actuator may be a pump button configured to actuation by a users thumb.

One embodiment of the sheath and balloon portion of the invention includes a flexible flap on the distal tip with an eyelet cut in the flap. This allows the invention to be anchored internally. The eyelet accommodates a suture or other means to tie onto the distal end of the device. The flexible flap can be pressed up against the abdominal surface for close fixation.

Preferably, the shaft comprises a lumen and at least one wire extending through the lumen, the wire having a distal end connected to a distal tip of the shaft, and a proximal end connected to the handle (or an actuator forming part of the handle), wherein movement of the handle/actuator relative to the shaft causes the distal end of the shaft to adjust between the substantially straight orientation and the curved orientation. Thus, tensioning the wire causes the distal end of the shaft to curve into a retraction position. Once tensioned, locking means can be employed to lock the wire in a tensioned position/state. Releasing the tension on the wire causes the distal end of the shaft to return to a straight orientation.

Typically, the distal end of the shaft comprises a spring beam. This should be understood to mean a beam having sufficient elastic properties to allow the beam bend into the curved orientation and return to a substantially straight orientation with permanent deformation. Typically, the spring beam is made of metal, and ideally comprises a rectangular cross section. The metal may be, for example, stainless steel such as stainless steel 301, or carbon steel such as carbon steel CS80. Polymeric beams having the required elastic properties are also envisaged.

Suitably the proximal end of the shaft comprises a rigid, generally a rigid metal tube, for example a stainless steel tube. A proximal end of the spring beam is suitably connected to a distal end of the metal tube which in turn typically mates with the distal end of the control handle. The spring beam is generally made from a spring steel material and a wire extends from the handle (or actuator forming part of the handle) through the metal tube and alongside the spring beam and terminates at the distal end of the spring beam. By applying tension to the wire as the handle is actuated, the spring beam can alter from its self-forming straight position to a curved position. The arrangement of the spring beam, which is typically rectangular in cross section, limits the movement of the beam to one direction. The degree of curvature may be varied by the handle up to a fully curved position, creating a diameter of between 20 and 160 mm. The elastic nature of the spring beam ensures that the distal end of the device may return to the initial straight position once the tension in the wire has been relieved.

A further embodiment of the shaft comprises a plurality of linked sections that are hingedly connected end-on-end to allow the shaft curve in a single plane. This embodiment of the central shaft is typically made from stainless steel tubing which is connected at the proximal end to the control handle and has a number of links at the distal end connected in a linear assembly pivoting about hinged joints. Each of the links is generally made from tubular stainless steel and the wire extends throughout the shaft concluding at the distal end. By pulling this wire, the shaft can be altered from a straight position to a curved position. The arrangement of the hinges limits the movement of the links to one plane. At least three links are typically used to provide a smooth curvature with a diameter of generally between 20 and 160 mm.

A further embodiment of the central shaft is a number interlocking segments that may be articulated by pulling a number of controlling wires. Each segment is capable of individual movement and the distal end of one segment is nested in the proximal end of the subsequent segment. One or more wires are threaded throughout each segment which will have at least one lumen. Once these wires are loosened, the assembly of segments can be placed in a particular curvature. The wires are then pulled in tension and in doing so cause a friction grip between adjacent segments locking them in place. This facilitates the elimination of hinges as mentioned in the prior embodiments.

A further embodiment of the central shaft is a composite shaft with rigid hollow tubing connected at its proximal end to the control handle and flexible tubing at its distal end. Two to eight wires extend from the control handle to the distal end of the invention. By pulling on one or more wires at a time, the flexible distal end will curve in the direction on the tensioned wire. By having a number of different wires, the flexible end can curve about a number of different planes increasing the manoeuvrability of the device. The diameter of curvature can be between 20 and 160 mm.

A further embodiment of the central shaft is a single polymer construction with between two and five flexure hinges spaced equidistant near the distal end of the shaft and a lumen passing throughout the polymer shaft. A wire resides within the inner lumen and is tethered at the distal end of the shaft. The flexure hinges are positioned in a longitudinal array and all limit the motion of the shaft to the same plane. Once the wire is tensioned, the shaft curves about the flexure hinges forming a curved position. The proximal end of the shaft connects to the control handle

The retractor is preferably adapted to be operated with one hand. Typically, the handle comprises an actuator, which suitably comprises a thumb dial coupled to a driving bolt, wherein the wire is connected to the driving bolt, and wherein rotation of the thumb dial causes displacement of the driving bolt along a longitudinal axis of the shaft so as to increase or decrease tension on the wire. The degree of curvature of the shaft may be varied depending on the magnitude of the dial's rotation up to a fully curved position, preferably of between 20 and 160 mm in diameter. The handle may be used in either hand and is generally made from a plastic material. Preferably the handle includes a method to lock the dial in position, preventing dial slippage.

A further embodiment of the handle includes a spring loaded mechanism and an “L” shaped key to facilitate the locking mechanism. The handle alters the shape of the shaft from its self-forming straight position to a curved position and is spring loaded to return the device to its initial straight position. The handle is capable of securing the shape of the device in the curved position by engaging a locking mechanism. An “L” shaped key is cut into the internal surface of sliding section of the handle. This key traces along a protrusion from the handle core. Withdrawing the sliding portion of the handle and twisting it when the protrusion corresponds with the perpendicular L section of the key, locks the handle in position. The sliding portion is connected internally to the proximal end of the wire, hence as the sliding portion of the handle is withdrawn, so too is the wire. This results in curving the distal end of the device. Furthermore twisting the sliding portion of the device in the opposite direction returns the control handle to its original shape owing to the spring loaded nature of the assembly of the handle.

A further embodiment of the handle comprises a hole for receipt of the user's thumb. The actuator may also comprise two finger supports, for example two slots dimensioned for receipt of a user first and second fingers. As the actuator is withdrawn with the first and second fingers relative to the user's thumb, the wire is tensioned and the distal end of the device alters from a self-forming straight position to a curved position. This handle may be spring loaded and a latching mechanism secures the retractor position in place.

A further embodiment of the handle comprises a trigger mechanism. This mechanism may be actuated by engaging the trigger with the user's first through fourth fingers and squeezing the trigger closed relative to the user's palm. A cam or gear system may be integrated into the handle to reduce the exertion force and thus user fatigue.

Preferably, the retractor may be fixed in position relative to the patient. A clamping mechanism may accompany the invention which secures the retractor relative to the patient. The distal end of the clamp comprises a means of integrating with the shaft of the retractor, preferably a speed clamp which can quickly fasten to the proximal end of the shaft. The proximal end of the clamp may be fixed to the operating table or to an independent rigid structure.

A further embodiment of the clamping mechanism is a slidable quick release ring. This ring fits over the extruded tubing between the control handle and the surgical cannula and can slide along the central axis of the device. Once the retractor has been engaged, hooking the internal organs, the clamping mechanism is slid into contact with the instrument port and is locked in place via a quick release lever.

A third embodiment of the clamping mechanism is a spring loaded clip which clasps the shaft of the invention in position once the internal organs have been successfully retracted.

In another aspect, the invention provides a laparoscopic retractor comprising a shaft having a proximal and distal end, the distal end of the shaft being configured for adjustment from a substantially straight orientation to a substantially U-shaped curved orientation suitable for laparoscopic retraction, the proximal end of the shaft comprising a control handle operably connected to the distal end of the shaft for adjustment of the distal end from the substantially straight orientation to the curved orientation, wherein the proximal end of the shaft comprises a metal tubing having a lumen and the distal end of the shaft comprises a metal spring beam, and wherein a wire is provided having a distal end fixed to a distal end of the metal spring beam and a proximal end operably connected to the control handle, wherein actuation of the control handle to apply tension to the wire causes metal spring beam to adjust from the substantially straight orientation to the substantially U-shaped curved orientation suitable for laparoscopic retraction.

Typically, the control handle comprises an actuator, which suitably comprises a thumb dial coupled to a driving bolt, wherein the wire is connected to the driving bolt, and wherein rotation of the thumb dial causes displacement of the driving bolt along a longitudinal axis of the shaft so as to increase or decrease tension on the wire.

In another aspect, the invention provides a laparoscopic retractor comprising a shaft having a proximal and distal end, the distal end of the shaft being configured for adjustment from a substantially straight orientation to a substantially U-shaped curved orientation suitable for laparoscopic retraction, the proximal end of the shaft comprising a control handle operably connected to the distal end of the shaft by means of a wire for adjustment of the distal end from the substantially straight orientation to the curved orientation, wherein the control handle comprises an actuator comprising a thumb dial coupled to a driving bolt, wherein the wire is connected to the driving bolt, and wherein rotation of the thumb dial causes displacement of the driving bolt along a longtitudinal axis of the shaft so as to increase or decrease tension on the wire.

The invention may also provide a kit suitable for performing laparoscopic or robotic abdominal surgery, the kit comprising a laparoscopic retractor according to the invention and an inflation device for inflating the tube and balloon(s) forming part of the laparoscopic retractor.

The invention also provides a method for performing laparoscopic or robotic abdominal surgery on a mammal comprising the steps of:
providing a laparoscopic retractor according to the invention having the distal end of the shaft in a substantially straight orientation;
making an incision in the abdomen of the individual;
inserting a trocar or cannula in the incision;
threading the laparoscopic retractor through the trocar or cannula and positioning the distal end of the shaft at a desired location;
inflating at least one balloon;
operating the actuator to adjust the distal end of the shaft to a curved orientation thereby performing laparoscopic retraction;
typically withdrawing the retractor slightly pulling the retracted organs out of the operating field and locking the retractor in position, and
performing laparoscopic or robotic abdominal surgery.

### BRIEF DESCRIPTION OF THE FIGURES

Other aspects, advantages and novel features of this invention will become more apparent from the following detailed description considered in connection with the accompanying drawings. However it should be understood that the drawings are designed as an illustration only and not as a definition of the limits of the invention. In the drawings:

FIG. 1 shows a perspective view of the retractor in the initial straight and deflated position;

FIG. 2 shows a perspective view of the retractor in the curved and inflated position;

FIG. 3 shows a front cross-sectional view of the retractor in the straight and inflated position;

FIG. 4 shows a top cross-sectional view of the retractor in the straight and inflated position;

FIG. 5 shows a top cross-sectional view of the retractor in the curved and inflated position;

FIG. 6 shows a perspective view of one embodiment of the invention with an alternative control handle with three finger slots and a clamping mechanism proximal to a surgical cannula;

FIG. 7 shows a perspective view of a second embodiment of the invention with an alternative control handle and smaller modular balloons;

FIG. 8 shows a perspective view of a third embodiment of the invention in the straight position with one long spherical balloon inflated by a syringe via a non-return valve;

FIG. 9 shows a perspective view of a fourth possible embodiment of the invention with an alternative handle and a clamping mechanism proximally located to a surgical cannula;

FIG. 10 shows an embodiment of the invention whereby it may be adapted for robotic control;

FIG. 11 shows a sketch of the retractor inserted through a surgical cannula in the abdominal space; and

FIG. 12 is a detailed view of the shaft of a laparoscopic retractor according to the invention.

### DETAILED DESCRIPTION OF THE PREFERRED EMBODIMENT

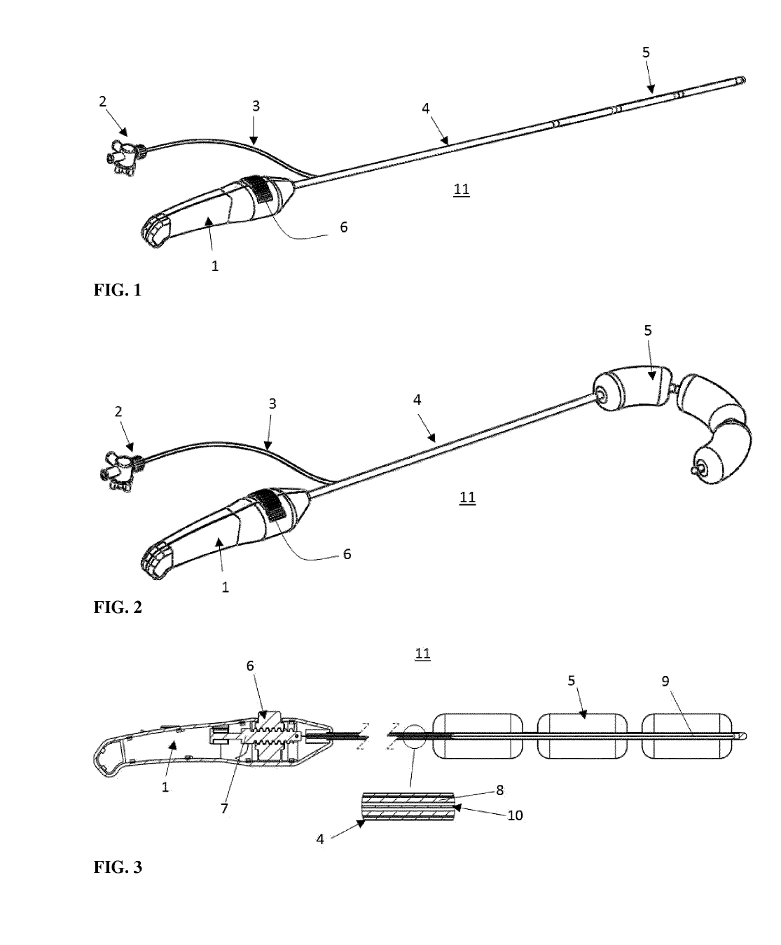

In the preferred embodiment of FIG. 1 and FIG. 2, the retractor 11 comprises of a control handle 1 connected to a tubular shaft which is covered by an extruded polymeric sheath 4 which has a number of balloons 5 fixed in a linear arrangement near the distal end of the device. The handle 1 comprises a thumb dial 6 used to actuate the device. By rotating the dial 6 with one's thumb, the user can alter the shape of the distal end of the device from the straight position to a curved orientation. A stop-cock 2 located at the proximal end of the inflation tubing 3 facilitates the inflation of the balloons 5. FIG. 1 shows the retractor 11 in its initial straight and deflated position. FIG. 2 shows the retractor 11 in its inflated curved position. The thumb dial 6 of the handle 1 has been rotated and in doing so pulls an internal wire which causes the distal portion of the device to curve. The diameter of curvature may extend from 20 to 160 mm.

A detailed view of the retractor 11 can be seen in the cross-section view in FIG. 3. The control handle 1 comprises a thumb dial 6 which is used to axially transfer a displacement bolt 7 which in turn is connected distally to an internal wire 10. The wire 10 passes through the central shaft 8 and terminates at the distal end of the spring beam 9. As the displacement bolt 7 is displaced axially, tension is applied to the internal wire 10 and in doing so causes the spring beam 9 to curve.

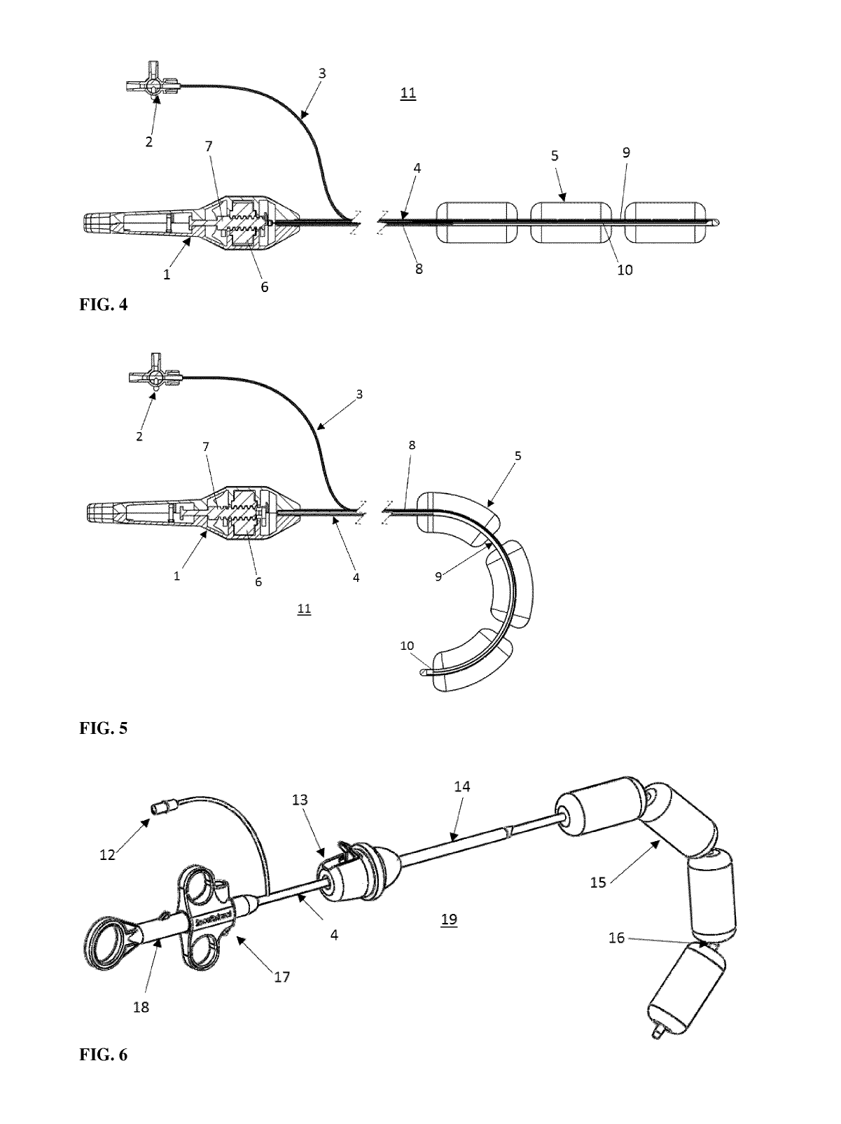

A top cross-sectional view of the retractor 11 can be seen in FIG. 4. As with FIG. 3, FIG. 4 illustrates the inner components of the retractor 11 in the straight and inflated position. In this embodiment, the inflation tubing 3 merges with the proximal end of a dual-lumen extruded tubing 4. The central lumen within the extruded tubing accommodates the central shaft 8 and spring beam 9 whilst a second lumen is used to inflate the modular balloons 5 mounted towards the distal end of the device. A stop-cock 2 fixed to the proximal end of the inflation tubing 3 prevents the balloons from deflating during operation. A non-return valve may also be used instead of the stop-cock 2.

FIG. 5 shows a top cross-sectional view of retractor 11 in the curved and inflated position. In this figure the thumb dial 6 has been fully engaged displacing the distal tip of the device by 180 degrees. The degree of curvature may vary depending on the magnitude of rotation of the dial 6. The distal end of the tubing 4 is sealed to prevent leakage once positioned within the peritoneal cavity.

A further embodiment of the retractor can be seen in FIG. 6 which comprises a handle 11 connected to a tubular shaft which is covered by an extruded tubing 4 which has a number of balloons 15 fixed in a linear arrangement near the distal end of the device. The handle comprises a central core 18 with a thumb support at the proximal end to improve usability of the device. A sliding portion 17 of the handle can be easily withdrawn to alter the shape of the device. A one-way valve 12 taps into the inflation lumen of the tubing 4 and facilitates the inflation of the balloons 15. In this embodiment 19 the shaft comprises a number of links 16 arranged linearly at the distal end of the shaft. As the sliding portion of the handle 17 is withdrawn, the internal wire curves the distal end of the device and in doing so triggers a spring loaded locking mechanism securing the device in its curved position. The diameter of curvature may extend from 20 to 160 mm.

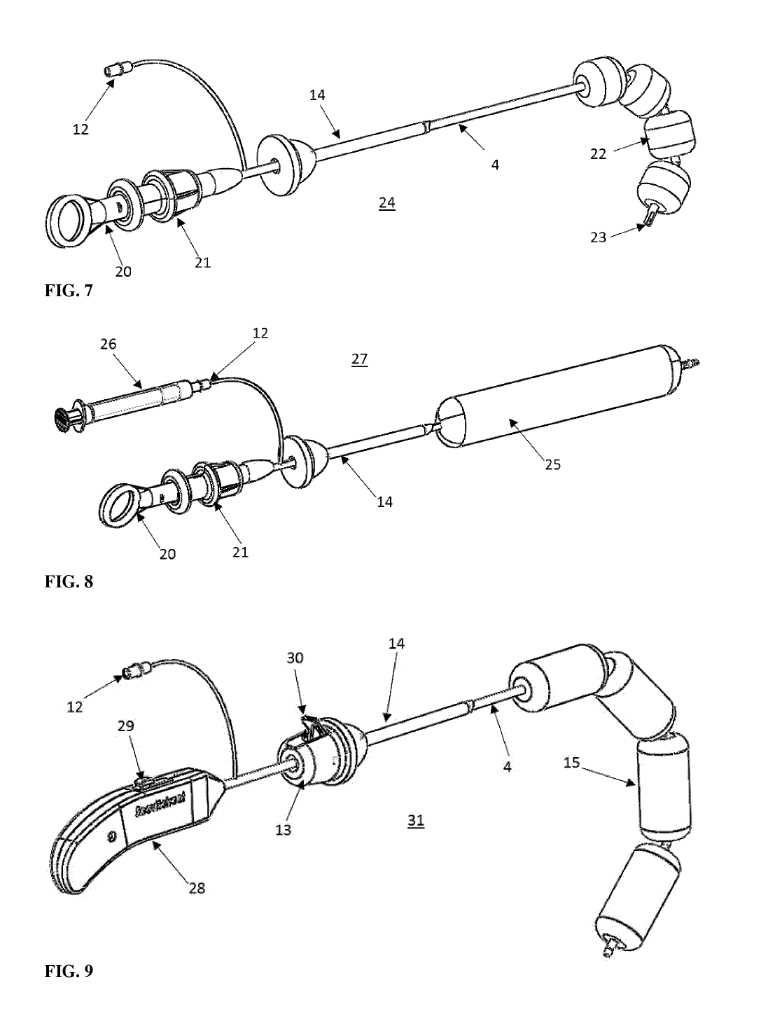

FIG. 7 shows one embodiment of the device 24 with an alternative handle. This handle 20 operates with a spring loaded “L” shaped locking mechanism and curves the balloons 22 and the shaft of the device once engaged. A surgical cannula or trocar 14 is pictured which acts as the access port for the retractor 24 to enter the abdominal space. As in FIG. 6, the shaft in this embodiment comprises a number of linearly arranged linkages. An eyelet 23 is located at the distal tip of the device to anchor the distal end of the device to an internal structure if required.

FIG. 8 shows a similar embodiment of the retractor depicted in FIG. 7, however in this embodiment the retractor 27 comprises a single long balloon 25. The balloon 25 is inflated through a non-return luer lock valve 12 by means of a syringe 26.

A further embodiment of the retractor can be seen in FIG. 9. In this embodiment 31, a thumb scroll 29 on the handle 28 is used to curve the shaft of the device by withdrawing an internal wire which terminates at the distal tip. FIG. 9 depicts a clamping mechanism 13 which prevents axial movement once the retractor has been positioned and is located proximally to the surgical cannula 14. The shaft of the retractor 4 passes through a channel in the main body of the clamp 13. A lever 30 which reduces the diameter of the channel is then latched in place clamping the retractor.

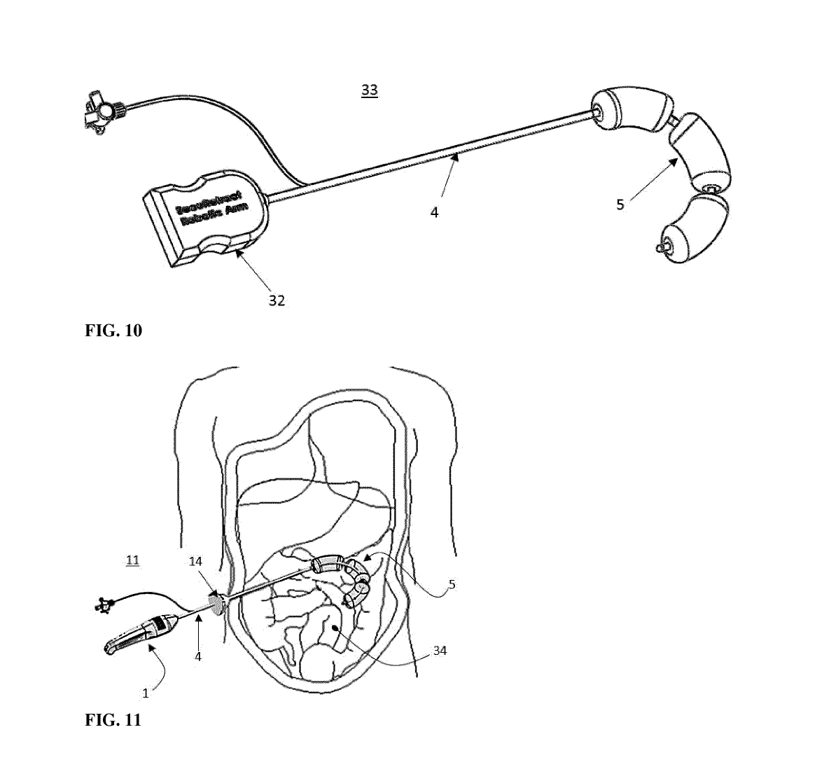

The retractor may also be adapted for use in robotic surgery. FIG. 10 shows the shaft 4 and balloons 5 of the retractor according to the invention, which are controlled by a mechanism 32 capable of integrating with a robotic interface. The movement and actuation of the shaft may be manipulated via robotic control.

FIG. 11 shows the preferred retractor 11 positioned within the abdominal space 34. The retractor 11 is inserted through a surgical cannula 14 and curved to retract the internal organs.

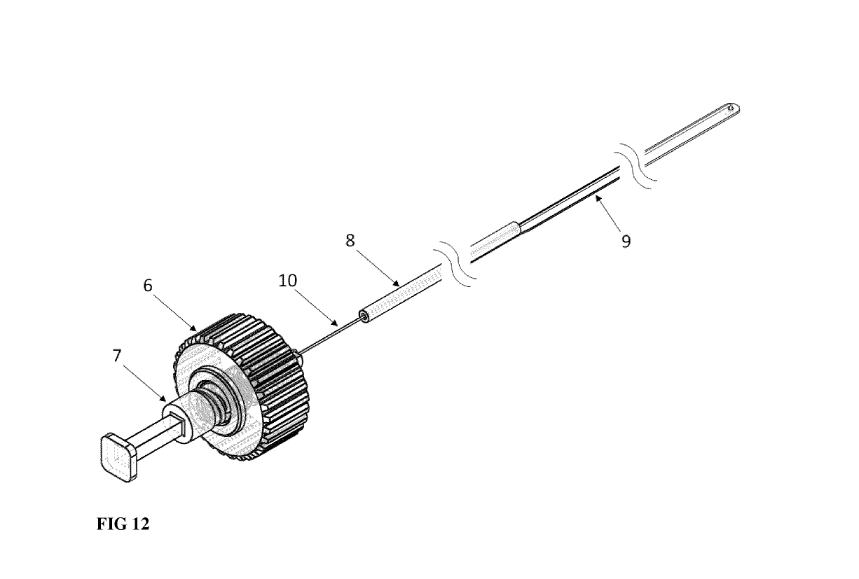

FIG. 12 is an illustration of the shaft 8 showing the tubular steel proximal section 40 fixed to the spring blade distal section 9, and the wire 10 projecting through the tubular steel section 40 and alongside the spring blade 9 and fixed to a distal end of the spring blade 9. Although not shown, the proximal end of the wire is attached to the displacement bolt 7 part of the actuator located within the handle 1.

The invention is not limited to the embodiments hereinbefore described which may be varied in construction and detail without departing from the spirit of the invention.

1. A laparoscopic retractor for atraumatic retraction in abdominal surgery comprising a shaft having a proximal and distal end, the distal end of the shaft being configured for adjustment from a substantially straight orientation to a curved orientation having a substantially U-shaped hook suitable for laparoscopic retraction, the proximal end of the shaft comprising a handle operably connected to the distal end of the shaft for adjustment of the distal end from the substantially straight orientation to the curved orientation, wherein the distal end of the shaft is enclosed within a flexible sheath that is adapted to be inflated, and wherein a portion of the sheath covering the substantially U-shaped hook comprises inflatable balloon means.

2. A laparoscopic retractor as claimed in claim 1 in which the inflatable balloon means extends along the length of the substantially U-shaped hook.

3. A laparoscopic retractor as claimed in claim 1 in which the balloon means comprises a plurality of inflatable balloons arranged axially along the sheath such that the inflatable balloons substantially cover the length of the substantially U-shaped hook.

4. A laparoscopic retractor as claimed in claim 1 in which the balloon means comprises at least three inflatable balloons arranged axially along the sheath.

5. A laparoscopic retractor as claimed in claim 1 in which the balloon means comprises a single inflatable elongated balloon arranged axially along the sheath.

6. A laparoscopic retractor as claimed in claim 1 in which the portion of the flexible sheath covering the substantially U-shaped hook is sealed at the distal end to prevent leakage once intracorporeally positioned.

7.-14. (canceled)

15. A laparoscopic retractor as claimed in claim 1, in which the distal end of the shaft is adjustable to a curved orientation having a diameter of curvature of between 20 and 160 mm.

16.-20. (canceled)

21. A laparoscopic retractor as claimed in claim 1 in which the distal end of the shaft comprises a plurality of linked sections that are connected by means of hinges end-on-end to allow the shaft curve in a single plane.

22. A laparoscopic retractor as claimed in claim 1 in which the handle comprises one or more formation configured to receive a user's hand, fingers or thumb.

23. A laparoscopic retractor as claimed in claim 22 in which the formations are selected from a trigger mechanism, a threaded dial mechanism or a mechanism which may be engaged by withdrawal of the first and second figures.

24. A laparoscopic retractor as claimed in claim 1 in which the inflatable balloon means comprises at least one and no more than five elongated tubular balloon mounted on the distal end of the shaft.

25. A laparoscopic retractor as claimed in claim 1 in which the or each inflatable balloon is cylindrical in shape with rounded edges with an axial length of 20-250 mm.

26. A laparoscopic retractor as claimed in claim 1 and in which the or each inflatable balloon has a cross-sectional diameter of 10-60 mm.

27. A laparoscopic retractor as claimed in claim 1, in which the inflatable balloon means is inflated through a common inflation channel or separate independent channels.

28. A laparoscopic retractor as claimed in claim 1, in which the inflatable balloon means comprise either dilation balloons or are made of a elastomeric material.

29.-32. (canceled)

33. A kit suitable for performing laparoscopic or robotic abdominal surgery, the kit comprising a laparoscopic retractor according to claim 1, and an inflation syringe or other inflation device for inflation of the tube and or at least one balloon.

34. A robotic surgical apparatus comprising a laparoscopic retractor according to claim 1.

35. (canceled)
